(19) United States
(12) Patent Application Publication
(10) Pub. No.: US 2023/0122826 A1
(43) Pub. Date: Apr. 20, 2023

(54) CONTINUOUS NON-INVASIVE ANALYTE MEASUREMENT SYSTEM AND METHOD

(71) Applicant: Edwards Lifesciences Corporation, Irvine, CA (US)

(72) Inventor: Julio Agustin Isla Garcia, Irvine, CA (US)

(21) Appl. No.: 18/066,813

(22) Filed: Dec. 15, 2022

Related U.S. Application Data
(63) Continuation of application No. PCT/US2021/038847, filed on Jun. 24, 2021.

(51) Int. Cl. A61B 5/00; A61B 5/145

(52) U.S. Cl. CPC A61B 5/0095; A61B 5/145; A61B 2562/028

(57) ABSTRACT

A system and method for non-invasively measuring at least one analyte within a blood vessel is provided. The system includes an excitation light source having at least one excitation laser configured to selectively produce an excitation light beam, an interrogation light source having at least interrogation laser configured to selectively produce an interrogation light beam at a predetermined interrogation wavelength, a Fabry-Perot sensor configured to be transparent to excitation light, and to reflect interrogation light, at least one light beam steering device, a light detector, and a controller in communication with the excitation light source, the interrogation light source, the at least one light beam steering device, the light detector, and a memory storing instructions.

This application claims priority to PCT/US2021/038847 filed Jun. 24, 2021, which claims priority to U.S. Patent Application Ser. No. 63/046,825 filed Jul. 1, 2020, the entireties of each of which are hereby incorporated herein by reference.

### BACKGROUND OF THE INVENTION

### 1. Technical Field

The present disclosure relates to systems and methods for non-invasively measuring analytes within mammalian blood vessels in general, and to systems and methods for non-invasively measuring analytes within mammalian blood vessels that use photometric and acoustics means in particular.

### 2. Background Information

Healthcare providers often monitor one or more physiological characteristics of a patient during diagnosis and treatment. A wide variety of monitoring devices have been developed to meet this need and have become an indispensable part of modern medicine. For example, it is known that determining the concentration of certain blood analytes (e.g., oxyhemoglobin, deoxyhemoglobin, etc.) within a subject's blood can facilitate the determination of clinical parameters (e.g., oxygen saturation, etc.), which in turn can provide insight into the subject's respiratory and/or cardiac function. Deviation from normal or expected values may alert a clinician to the presence of a particular physiologic condition. Some known monitoring techniques require an invasive arterial catheter cannulated into a subject's arterial bloodstream. Such invasiveness techniques may cause the patient discomfort, injury, and/or inconvenience. Hence, it would be desirable to provide a non-invasive system and/or method operable to measure analytes within the blood vessels of a subject.

### SUMMARY

According to an aspect of the present disclosure, a system for non-invasively measuring at least one analyte within a blood vessel is provided. The system includes an excitation light source, an interrogation light source, a Fabry-Perot sensor, at least one light beam steering device, a light detector, and a controller. The excitation light source has at least one excitation laser configured to selectively produce an excitation light beam at a predetermined excitation wavelength. Absorption by the analyte of an amount of light at the excitation wavelength causes the analyte to produce a photoacoustic response. The interrogation light source has at least one interrogation laser configured to selectively produce an interrogation light beam at a predetermined interrogation wavelength. The Fabry-Perot sensor is configured to be transparent to light at the excitation wavelength, and to reflect light at the interrogation wavelength. The light detector is operable to receive light reflected from the Fabry-Perot sensor and produce signals representative of the received light. The controller is in communication with the excitation light source, the interrogation light source, the at least one light beam steering device, the light detector, and a memory storing instructions. The instructions when executed cause the controller to control the light beam steering device to steer both the excitation light beam and the interrogation light beam relative to the Fabry-Perot sensor, and to measure an amount of the analyte within the blood vessel using the signals representative of the received light.

In any of the aspects or embodiments described above and herein, the system may be configured to steer the excitation light beam and the interrogation light beam in unison in a direction of travel along a path relative to the Fabry-Perot sensor. The path may be a Lissajous pattern.

In any of the aspects or embodiments described above and herein, the light beam steering device may include a two-axis micro-electro-mechanical (MEMS) mirror, and the instructions when executed may cause the controller to control the MEMS mirror to steer the excitation light beam and the interrogation light beam in unison.

In any of the aspects or embodiments described above and herein, the instructions when executed may cause the controller to control the two-axis MEMS mirror using a resonant excitation.

In any of the aspects or embodiments described above and herein, the system may further include a sensor head configured for attachment to a subject, and the two-axis MEMS mirror and the Fabry-Perot sensor are disposed within the sensor head.

In any of the aspects or embodiments described above and herein, the system may be configured to produce the excitation light beam and the interrogation light beam substantially coincide with one another in a sensing area of the Fabry-Perot sensor.

In any of the aspects or embodiments described above and herein, the system may be configured to produce the excitation light beam at a first position on the path, and produce the interrogation light beam at a second position on the path, the second position lagging behind the first position on the path in the direction of travel.

In any of the aspects or embodiments described above and herein, the light beam steering device may include a first two-axis micro-electro-mechanical (MEMS) mirror and a second two-axis MEMS mirror, and the instructions when executed may cause the controller to control the first MEMS mirror to steer the excitation light beam and to control the second MEMS mirror to steer the interrogation light beam in unison.

In any of the aspects or embodiments described above and herein, the Fabry-Perot sensor has a sensing area, and the system may be configured to produce the excitation light source to be incident to the Fabry-Perot sensor in an excitation incident area, and the excitation incident area is less than the sensing area.

In any of the aspects or embodiments described above and herein, the excitation light source may include a plurality of excitation lasers, each configured to selectively produce an excitation light beam at a unique excitation wavelength relative to the other excitation wavelengths of the plurality of excitation lasers. The controller may operate the excitation lasers sequentially.

In any of the aspects or embodiments described above and herein, the system may include one or more optical fibers in communication with the excitation light source, the optical fibers configured to accept light at a plurality of the excitation wavelengths.

In any of the aspects or embodiments described above and herein, the system may be configured so that the light reflected from the Fabry-Perot sensor is received by the one or more optical fibers and passed to a light detector, the light detector configured to produce the signals representative of the received light and communication the signals to controller.

In any of the aspects or embodiments described above and herein, the interrogation light source may include a plurality of interrogation lasers, each configured to selectively produce an interrogation light beam at a unique interrogation wavelength relative to the other interrogation wavelengths of the plurality of interrogation lasers. The controller may operate the plurality of interrogation lasers sequentially.

In any of the aspects or embodiments described above and herein, the system may include one or more optical fibers in communication with the interrogation light source, the optical fibers configured to accept light at a plurality of the interrogation wavelengths.

In any of the aspects or embodiments described above and herein, the Fabry-Perot sensor may include a plurality of alignment cells, each configured to provide position location information. Each alignment cell may be distinguishable from the other alignment cells by the position location information it is configured to provide. The alignment cells may be disposed outside of a sensing area of the Fabry-Perot sensor.

In any of the aspects or embodiments described above and herein, the instructions when executed may cause the controller to calibrate the Fabry-Perot sensor using a sensitivity map. The sensitivity map may be based on scans of the Fabry-Perot sensor using an interrogation light beam at one or more interrogation wavelengths.

In any of the aspects or embodiments described above and herein, the instructions when executed may cause the controller to create a vascular map of tissue being sensed with the excitation light beam, the vascular map including the location of blood vessels within the tissue. The vascular map may include the location of one or more veins in the tissue and one or more arteries within the tissue based on relative amounts different analytes sensed within the blood vessels.

According to another aspect of the present disclosure, a method of non-invasively measuring at least one analyte within a blood vessel is provided. The method includes: a) providing a system having an excitation light source with at least one excitation laser configured to selectively produce an excitation light beam at a predetermined excitation wavelength, an interrogation light source having at least interrogation laser configured to selectively produce an interrogation light beam at a predetermined interrogation wavelength, a Fabry-Perot sensor configured to be transparent to said light at the excitation wavelength, and to reflect said light at the interrogation wavelength, at least one light beam steering device, a light detector, and a controller; b) using the at least one light beam steering device to steer the excitation light beam and the interrogation light beam in unison in a direction of travel along a path relative to the Fabry-Perot sensor, wherein absorption by the analyte of an amount of light at the excitation wavelength causes the analyte to produce a photoacoustic response; c) receiving light reflected from the Fabry-Perot sensor, and using the light detector to produce signals representative of the received light and communicate the signals to the controller; and d) measuring an amount of the analyte within the blood vessel using the signals representative of the received light.

A further understanding of the nature and advantages of the present invention are set forth in the following description and claims and accompanying drawings.

### BRIEF DESCRIPTION OF THE DRAWINGS

FIG. 1 is a diagram of an embodiment of the present disclosure system.

FIG. 2 is a diagram of a sensor head embodiment of the present disclosure system in communication with a subject digit.

FIG. 2A is a diagram of a sensor head embodiment of the present disclosure system.

FIG. 3 is a diagram of an excitation light source embodiment.

FIG. 4 is a diagram of an interrogation light source embodiment.

FIG. 5 is a diagram of an interrogation light source embodiment.

FIG. 6 is a diagram of a light beam steering device control loop embodiment.

FIG. 6A is a diagram of a light beam steering device control loop embodiment.

FIG. 7 is a diagram illustrating an excitation light beam and an interrogation light beam substantially coincident with one another, and a steering pattern relative to a Fabry-Perot sensor.

FIG. 8 is a diagram illustrating an excitation light beam and an interrogation light beam substantially coincident with one another, and a Lissajous steering pattern relative to a Fabry-Perot sensor.

FIG. 9 is a diagram illustrating an incident light beam configuration wherein the interrogation light beam lags behind the excitation light beam in a direction of travel.

FIG. 10 is a diagram illustrating an interrogation light beam lagging behind an excitation light beam in a direction of travel along a Lissajous steering pattern relative to a Fabry-Perot sensor.

FIG. 11 is a diagram illustrating an interrogation light beam steering pattern relative to a Fabry-Perot sensor, and a sensitivity map representative of the interrogation light beam engagement with the Fabry-Perot sensor.

FIG. 12 is a flow chart of an exemplary calibrating procedure.

FIG. 13 is a diagram of method steps for producing a vasculature map of vessels within subject tissue according to the present disclosure.

### DETAILED DESCRIPTION

The present disclosure is directed to a system and method for continuously and non-invasively measuring analytes within blood vessels. As used herein, the term “blood vessel” is intended to include veins, arteries, or capillaries of a subject, or any combination thereof. The present disclosure is operable to measure a plurality of different analytes, including oxyhemoglobin (“HbO2”) and deoxyhemoglobin (“Hb”) in blood vessels. Hemoglobin (in both oxygenated and deoxygenated states) has a substantially high concentration within a blood sample, and has a substantially high absorption coefficient. The present disclosure is, therefore, well suited to sensing and measuring hemoglobin. The present disclosure is not, however, limited to sensing and measuring Hb and HbO2. Additional non-limiting examples of analytes that may be measured include endogenous analytes such as methemoglobin, carboxyhemoglobin, water, melanin, lipids content, and carbon monoxide, and exogenous analytes such as indocyanine green, methylene blue dye, and the like. Signal data collected using the present disclosure can be used to determine physiological/hemodynamic parameters, such as oxygen saturation, cardiac output, etc.

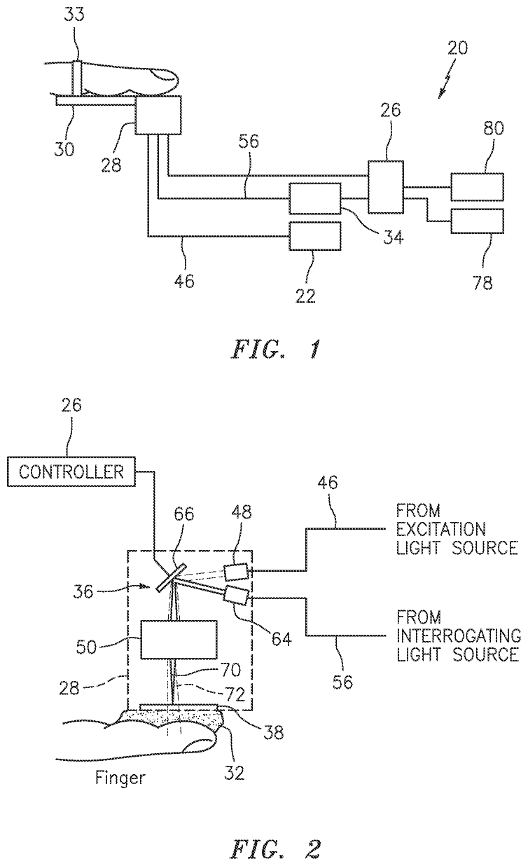

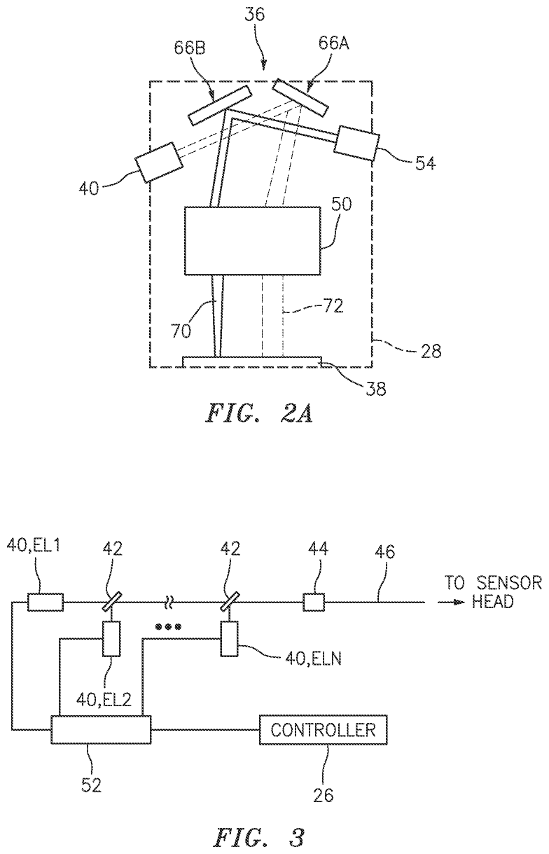

Referring to FIGS. 1 and 2, a system 20 according to present disclosure may include an excitation light source 22, an ultrasound receiving mechanism, a controller 26 in communication with the excitation light source 22 and the ultrasound receiving mechanism, and a sensor head 28 configured for attachment to a subject; e.g., to a digit of the subject.

The sensor head 28 is configured for attachment to a subject in a position where excitation light can access blood vessels containing analytes of interest. The sensor head 28 shown in FIG. 1 is configured for attachment to a subject's finger, including structure 30 for immobilizing the subject's finger. In FIG. 2, a couplant 32 is disposed between the sensor head 28 and the subject's digit. The present disclosure is not, however, limited to a sensor head 28 configured for attachment to a subject's digit. The sensor head 28 embodiment shown in FIG. 1 includes a strap 33 that is operable to secure the sensor head 28 to a subject's digit. The present disclosure is not limited to any particular means for securing the sensor head 28 to the subject. The sensor head 28 is configured to receive a laser light beam from the excitation light source 22 and one or more laser light beams from an interrogating light source 34. The sensor head 28 includes a light beam steering device 36 and a Fabry-Perot sensor 38 (see FIG. 2) described below as an element within the ultrasound receiving mechanism. In some embodiments, the sensor head 28 may also include elements operable to condition (e.g., collimate, focus, filter, etc.) the aforesaid laser light beams; e.g., collimating optics, objective optics, etc.

The excitation light source 22 is operable to produce light at a plurality of predetermined wavelengths: e.g., in a pulsed mode. In some embodiments, the excitation light source 22 includes a plurality of lasers (referred to hereinafter as “excitation lasers 40”), each configured to emit a light beam at a particular wavelength of light. Each excitation laser 40 may be configured to emit a light beam at a wavelength of light different from that emitted from the other excitation lasers 40. The wavelength of each excitation laser 40 is chosen based on its ability to produce a photoacoustic effect (sometimes referred to as an “optoacoustic effect”) when light emitted by the excitation laser 40 is sufficiently absorbed by a target analyte. The term “photoacoustic effect” as used herein refers to the phenomenon that occurs when light at a particular wavelength is presented to and absorbed by the target analyte, thereby causing an increase in kinetic energy of the target analyte and consequent pressure response from the analyte in the form of an acoustic wave.

In some embodiments, the excitation light source 22 is configured to produce an excitation light beam 72 that includes light produced by at least one of the excitation lasers 40. Optical beam combiners 42 (e.g., a dichroic mirror) may be used to add light from each respective excitation laser 40 into the excitation light beam 72 prior to the excitation light beam 72 being presented to the sensor head 28 for application to the subject. FIG. 3 diagrammatically illustrates a plurality of excitation lasers 40 (i.e., EL1, EL2 . . . ELN) each operable to emit a light beam at a respective wavelength (i.e., λ1, λ2 . . . λN), where “N” is an integer. One or more optical fibers (or other light conduits) may be used to conduct light within the structure for producing the excitation light beam 72. The excitation light beam 72 may be directed through optical fiber launching optics 44 (e.g., including one or more focusing lenses, an fc/pc connector, etc.) and into an optical fiber 46 (or other light conducting device) for passage to the sensor head 28. The optical fiber 46 providing the light conduit to the sensor head 28 may be configured to accept light at a plurality of different wavelengths. A non-limiting example of an optical fiber 46 configured to accept light at a plurality of different wavelengths is an endlessly single-mode photonic crystal fiber. As stated above, in some embodiments, the sensor head 28 may include collimating optics 48 in communication with the optical fiber 46 conducting the excitation light beam 72, and in communication with the objective optics 50 for conditioning the excitation light beam 72. FIG. 3 also illustrates a laser control unit 52 for controlling the operation of the plurality of excitation lasers 40; e.g., for synchronized firing of the excitation lasers 40. The laser control unit may be independent of and in communication with the system controller 26, or may be integrated with the system controller 26.

In some embodiments, the ultrasound receiving mechanism includes a Fabry-Perot sensor 38 and an interrogation light source 34 having at least one interrogation laser 54. FIG. 2 diagrammatically illustrates a sensor head 28 embodiment that includes the Fabry-Perot sensor 38 portion of the ultrasound receiving mechanism.

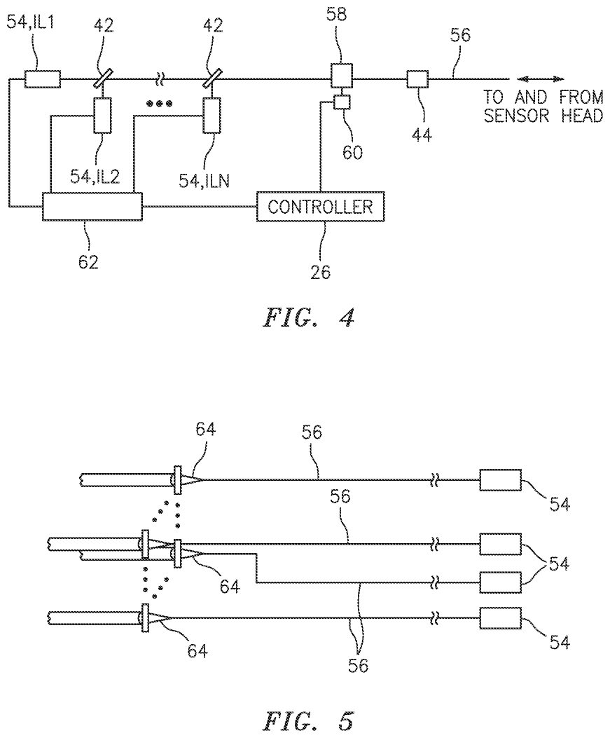

In some embodiments, the interrogation light source 34 includes a plurality of interrogation lasers 54. As will be explained herein, using a plurality of interrogation lasers 54, each emitting at light beam at a different wavelength, may provide an improved ability to detect deformations of the Fabry-Perot sensor 38, and therefore acoustic waves. The interrogation light source 34 is configured to produce an interrogation light beam 70 that includes light produced by at least one of the interrogation lasers 54. Optical beam combiners 42 may be used to add light from each respective interrogation laser 54 into the interrogation light beam 70. FIG. 4 diagrammatically illustrates a plurality of interrogation lasers 54 (i.e., IL1, IL2 . . . ILN) each operable to emit a light beam at a respective wavelength (i.e., λ1, λ2 . . . λN), where “N” in an integer. One or more optical fibers (or other light conduit) may be used to conduct light within the structure for producing the interrogation light beam. The interrogation light beam 70 may be directed through optical fiber launching optics 44 (e.g., including one or more focusing lenses, an fc/pc connector, etc.) and into an optical fiber 56 (or other light conducting device) for passage to the sensor head 28. The optical fiber 56 providing the light conduit to the sensor head 28 may be configured to accept light at a plurality of different wavelengths. As indicated above, a non-limiting example of an optical fiber 56 configured to accept light at a plurality of wavelengths is an endlessly single-mode photonic crystal fiber. FIG. 4 illustrates a beam splitter 58 disposed to allow the interrogation light beam 70 to pass through to the optical fiber launching optics 44 and subsequently to the sensor head 28. A light detector 60 (e.g., having one or more photodiodes) is in communication with the beam splitter 58. As stated above, in some embodiments, the sensor head 28 may include collimating optics 64 in communication with the optical fiber 56 conducting the interrogation light beam 70, and in communication with the objective optics 50 for conditioning the interrogating light beam 70. As will be described below, light collected from the sensor head 28 (e.g., light emanating from a Fabry-Perot sensor 38) is permitted to pass through the optical fiber 56 providing the light conduit between the interrogation light source 34 and the sensor head 28. The beam splitter 58 is operable to direct such collected light to the light detector 60. The present disclosure is not limited to any particular type of beam splitter 58; e.g., a dichroic mirror, a fiber-based light splitting mechanism, etc. FIG. 4 also illustrates a laser control unit 62 for controlling the operation of the plurality of interrogation lasers 54; e.g., synchronized firing of the interrogation lasers 54. The laser control unit 62 may be independent of and in communication with the system controller 26, or may be integrated with the system controller 26.

Referring to FIG. 5, in some embodiments the ultrasound receiving mechanism is configured to include a plurality of interrogation lasers 54 that are collectively applied to the Fabry-Perot sensor 38 to detect deformations of the Fabry-Perot sensor 38, and therefore acoustic waves. The light beams from the plurality of interrogation lasers 54 may be configured in parallel (e.g., using collimating optic devices). The parallel interrogation light beams encompass a greater area than that of a single interrogation light beam, and therefore may facilitate detection of Fabry-Perot sensor 38 deformations and the speed at which the sensor may be scanned. As indicated above, the plurality of interrogation lasers 54 (i.e., IL1, IL2 . . . ILN) may each emit a light beam at a different respective wavelength (i.e., λ1, λ2 . . . λN).

The light beam steering device 36 is operable to: a) receive input light beams from the excitation light source 22 (e.g., the excitation light beam 70) and the interrogation light source 34 (e.g., the interrogation light beam 72 or the plurality of parallel interrogation light beams), and to direct those light beams toward the Fabry-Perot sensor 38 and subject tissue; and b) to steer the aforesaid input light beams 70, 72 in unison in a predetermined pattern relative to the Fabry-Perot sensor 38 to permit light beam incidence of a select portion, or all, of the Fabry-Perot sensor 38. As explained below, the light beam steering device 36 of the present disclosure enables analyte sensing with an excitation light beam 72 presented to the tissue in a manner that facilitates meeting the requisite safety regulations, while at the same time provided an improved means for determining analyte information. The light beam steering device 36 is in communication with the controller 26 and the controller 26 may be configured to control the light beam steering device 36 as will be described below.

An example of an acceptable light beam steering device 36 is a two-axis micro-electro-mechanical mirror (referred to hereinafter as a “MEMS mirror 66”). Briefly stated, a two axis MEMS mirror 66 includes a mirror that can be controllably pivoted along two orthogonal axes. The reflection of a light beam from the mirror can therefore be controlled by selectively pivoting the mirror. FIG. 2 illustrates a sensor head 28 embodiment having light beam steering device 36 that includes a single two axis MEMS mirror 66 configured to receive input excitation and interrogation light beams 72, 70. FIG. 2A illustrates an alternative sensor head 28 embodiment having light beam steering device 36 that includes a first two axis MEMS mirror 66A configured to receive an input excitation light beam 72 and a second two axis MEMS mirror 66B configured to receive an input interrogation light beam 70. A person of skill in the art will recognize that two axis MEMS mirrors, and control logic for the same, are known and no further detail is required for an enabling description herein. In those embodiments wherein a plurality of interrogation light beams 70 are collectively applied to the Fabry-Perot sensor 38, an array of two-axis MEMS mirrors may be used to steer the aforesaid light beams. A two axis MEMS mirror is a preferred example of a light beam steering device 36 because, for example, it is lightweight and compact relative to other light beam steering devices currently available, and because it can be operated at a rapid scanning speed compared to currently available devices. The present disclosure is not limited however, to using a MEMS mirror.

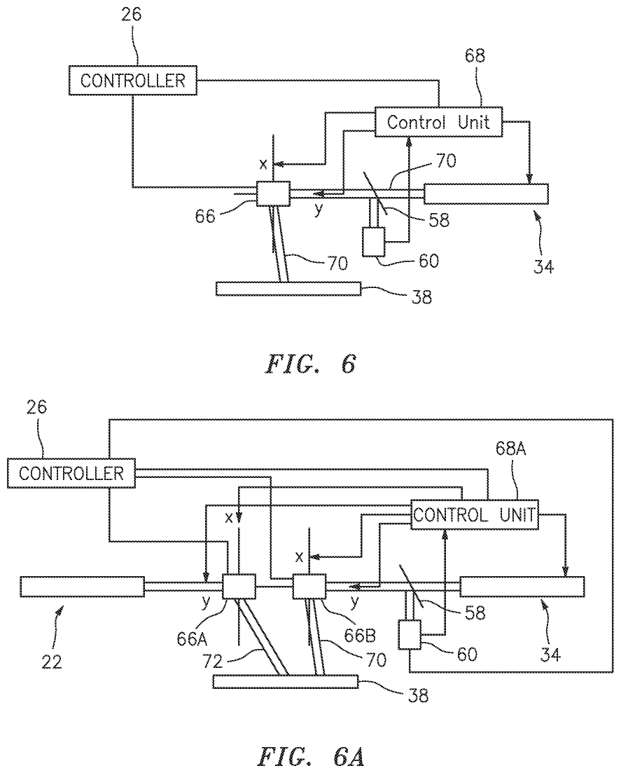

FIG. 6 illustrates a control loop that may be used for a single two axis MEMS mirror 66 (e.g., see FIG. 2). The control unit 68 shown in FIG. 6 (which may be integral to the controller 26, or may be independent of, but in communication with, the controller 26) is in communication with the interrogation light source 34, the light detector 60, and the MEMS mirror 66. As is explained herein, the interrogation light source 34 produces an interrogation light beam 70 that is directed to the Fabry-Perot sensor 38 by the MEMS mirror 66. Light reflected from the Fabry-Perot sensor 38 is collected and passed to the optical conduit 56 (e.g., see FIG. 2) between the sensor head 28 and the interrogation light source 34. A beam splitter 58 directs the collected light to the light detector 60, which in turn produces signals representative of the collected light.

FIG. 6A illustrates a control loop that may be used for a pair of two axis MEMS mirrors 66A, 66B (e.g., see FIG. 2A). The control unit 68A shown in FIG. 6A (which may be integral to the controller 26, or may be independent of, but in communication with, the controller 26) is in communication with the interrogation light source 34, the light detector 60, the first MEMS mirror 66A, and the second MEMS mirror 66B. This control loop is similar in function to that described above with regard to the single MEMS mirror 66 embodiment.

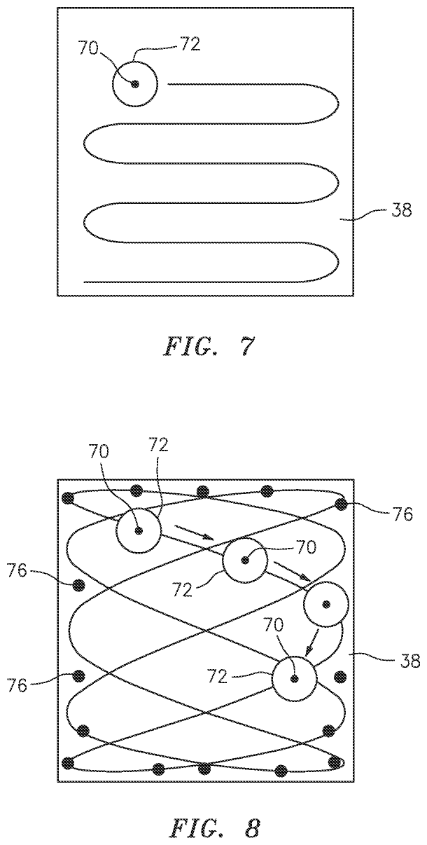

In some embodiments, the excitation and interrogation light beams 72, 70 are input into the light beam steering device 36 so that the aforesaid light beams are reflected from the light beam steering device 36 in a substantially coincident manner; e.g., see FIGS. 2, 7, and 8. The diagram shown in FIG. 7 diagrammatically illustrates an embodiment where an excitation beam 72 and an interrogation beam 70 are substantially coincident as they are steered (e.g., in a raster pattern) relative to the Fabry-Perot sensor 38. FIG. 8 diagrammatically illustrates an embodiment where an excitation beam 72 and an interrogation beam 70 are substantially coincident as they are steered (e.g., in a Lissajous pattern) relative to the Fabry-Perot sensor 38. In these embodiments, the footprint of the excitation light beam 72, as projected onto the tissue, encompasses the area of the Fabry-Perot sensor 38 that is being read with the interrogating light beam 70. The light beam steering device 36 may, therefore, be used to simultaneously steer the aforesaid substantially coincident light beams 70, 72. In contrast with sensing systems that interrogate an entire region of tissue with excitation light while sensing only a small portion of the same region, this embodiment of the present disclosure limits the excitation light exposure of the tissue to only that portion of tissue that is concurrently being sensed for a response to the excitation light. This aspect of the present disclosure is particularly beneficial in those instances where the tissue is being subjected to a substantial intensity of excitation light. A person of skill will recognize that safety regulations typically limit the total amount of energy to which tissue can be exposed (via the excitation light), and/or the intensity level of light to which the tissue can be exposed in a given period of time. Embodiments of the present disclosure permit blood analyte sensing with a lower tissue energy exposure than current systems of which we are aware, typically permit use of lower intensity excitation light beams thereby improving subject safety, and typically improve signal-to-noise ratio since excitation energy is not applied to areas that are not being sensed, in contrast to existing systems.

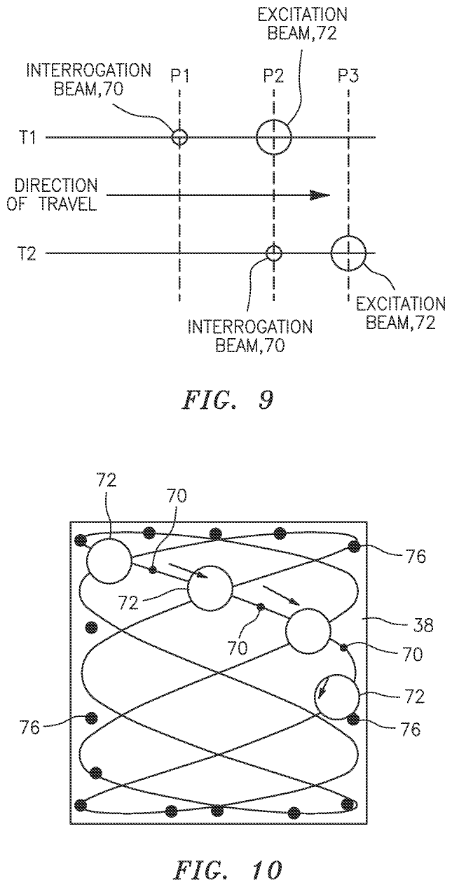

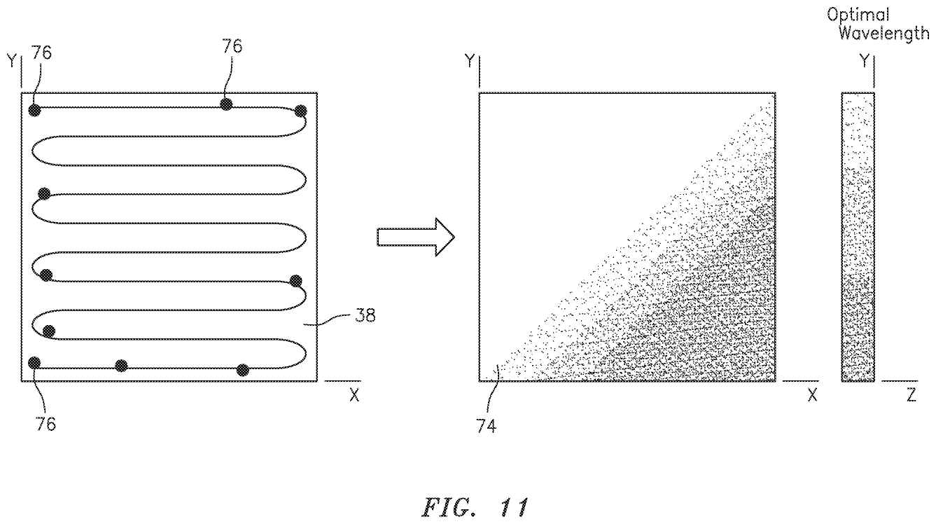

In some embodiments, the light beam steering device 36 is configured so that the interrogation beam 70 and the excitation light beam 72 are not substantially coincident, but are steered together. For example, the interrogation beam 70 may be oriented to positionally lag behind the excitation beam 72. Referring to FIG. 9, a time/position diagram is shown. At a first point in time (“T1”), the interrogation beam 70 is located at a first position (“P1”) and the excitation light beam 72 is located at a second position (“P2”), forward of the interrogation beam 70 in the direction of travel. At a later point in time (“T2”), the interrogation beam 70 is located at the second position P2 and the excitation light beam 72 is located at a third position (“P3”), forward of the second position P2 in the direction of travel. FIG. 10 diagrammatically illustrates an embodiment where the interrogation beam 70 lags behind the excitation beam 72 as they are steered (e.g., in a Lissajous pattern) relative to the Fabry-Perot sensor 38. The configuration diagrammatically depicted in FIGS. 9 and 10 wherein the interrogation beam 70 is incident on a region of the Fabry-Perot sensor 38 independent of the region where the excitation beam 72 is incident, is a non-limiting example. In alternative embodiments, the excitation beam 72 and interrogation beam 70 may overlap one another to some degree, but not be substantially coincident. Hence as the light beams 70, 72 are steered together, the interrogation beam 70 lags to some degree behind the excitation beam 72 along the direction of travel. Configurations wherein the interrogation light beam 70 positionally lag the excitation light beam 72 are understood to receive ultrasonic signals from target analyte disposed deeper within the subject's tissue. Hence, these configurations may enable monitoring with greater sensitivity and/or at deeper depths from the skin surface of the subject.

As stated above, the light beam steering device 36 can be used to steer the excitation and interrogation light beams 72, 70 in a predetermined pattern relative to the Fabry-Perot sensor 38 to permit light beam incidence of a select portion, or all, of the Fabry-Perot sensor 38. In some embodiments the two axis MEMS mirror 66 can be controlled to steer the excitation and interrogating laser beams 72, 70 in a Lissajous pattern; e.g., see FIGS. 8 and 10. A benefit of using a Lissajous steering pattern is that both dimensions of the two-axis MEMS mirror 66 can be controlled using a resonant excitation. A resonant excitation may allow for a substantially faster scanning speed compared to, for example, controlling the same mirror using non-resonant excitation. The excitation (at least frequency and phase) of both dimensions is set such that substantial scanning spatial resolution is achieved. The present disclosure is not, however, limited to steering the excitation and interrogation light beams 72, 70 in a Lissajous pattern. Alternative steering configurations include rastering, grid scanning, or any other pattern that permits the present disclosure system 20 to sense the tissue of interest.

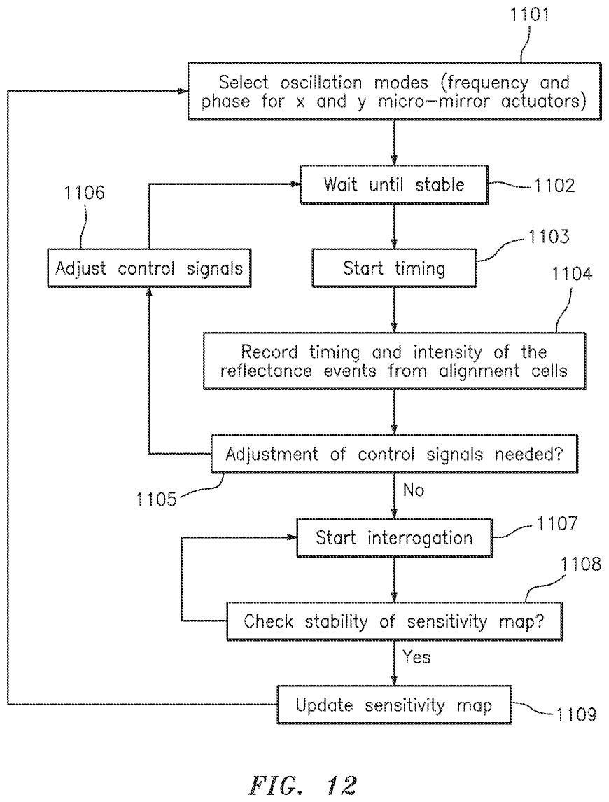

As will be explained below, in some embodiments the light beam steering device 36 may be used to steer the interrogation light beams to obtain a sensitivity map for the Fabry-Perot sensor 38 (e.g., see FIG. 11) that is subsequently used to select an optical wavelength(s) of the interrogating light beam.

The Fabry-Perot sensor 38 is configured with internal surfaces that reflect light at select wavelengths, and configured to be transparent to light at other wavelengths. In the present disclosure, the Fabry-Perot sensor 38 is configured to be transparent to the excitation light beam 72 wavelengths and reflects the interrogation light beam 70 wavelengths. Hence, the excitation light beam 72 passes through the Fabry-Perot sensor 38 and is incident to the aligned tissue region. FIG. 2 illustrates this aspect of the present disclosure with the excitation light beam 72 and interrogation light beam 70 in a substantially coincident configuration. The region of the Fabry-Perot sensor 38 that is transparent to the excitation light beam 72 wavelength and reflects the interrogation light beam 70 wavelength may be referred to as the “sensing area” of the sensor 38. The Fabry-Perot sensor 38 may be described as being “deformable” meaning that the Fabry-Perot sensor 38 is configured to assume a “default” configuration in the absence of any external forces acting on it (e.g., external forces such as those caused by an acoustic wave), and may be changed to a plurality of alternative configurations (i.e., “deformed configurations”) when an external force (e.g., an acoustic wave) acts on the sensor 38. In the default configuration, the internal reflective surfaces may be substantially parallel, and disposed a predetermined distance from one another. Light reflected from the Fabry-Perot sensor 38 in the default configuration is consistently modified (e.g., phase change, intensity, etc.) to have one or more determinable photometric characteristics. To be clear, a Fabry-Perot sensor 38 when applied to a subject—but not yet subjected to acoustic waves resulting from analyte excitation—may not have parallel reflective surfaces; e.g., because of a force applied to the Fabry-Perot sensor 38, or because of environmental conditions (e.g., a temperature gradient), etc. In such an instance, the Fabry-Perot sensor 38 may still be considered to be in a “default” configuration due to the absence of acoustic waves acting on the sensor 38. A pressure wave of sufficient magnitude acting on the Fabry-Perot sensor 38 changes the relative positions of the internal reflective surfaces (i.e., deforms the sensor). Light reflected from the Fabry-Perot sensor 38 when the sensor 38 is in a deformed configuration is modified in a manner other than that associated with the default configuration (e.g., differences in phase change, intensity, etc.). Hence, a change in the reflected light characteristics indicates a force has been applied to the sensor 38. The degree to which reflected light characteristics are changed is a function of the magnitude of the force applied to the sensor 38. Hence, the Fabry-Perot sensor 38 as used in the present disclosure is operable to sense pressure/acoustic waves. In some embodiments, a Fabry-Perot sensor 38 may also be used to measure a temperature gradient due to thermal expansion coefficient, which temperature gradient in turn can be used for flow measurements. The configuration of the Fabry-Perot sensor 38 is chosen such that an acoustic wave of a type and magnitude produced by an analyte interrogated by an excitation wavelength as described herein, will produce a detectable difference in the reflected interrogating light. A Fabry-Perot sensor 38 as used in the present disclosure may be described, therefore, as converting received acoustic waves/pressure waves signal to optical signals, which optical signals may be detected by a light detector 60. The Fabry-Perot sensor 38 as used in the present disclosure simplifies the ultrasound receiving mechanism by utilizing fewer reading channels (e.g., counted as the total number of optical fibers or electrical wires) in contrast to, for example, an ultrasound array composed of piezoelectric elements or elements that produce an electrical signal when mechanically deformed.

In some embodiments, the Fabry-Perot sensor 38 may include reflectance-encoded alignment cells 76 (e.g., see FIGS. 8, 10, and 11) that can be used to align the impinging light beams relative to the Fabry-Perot sensor 38. The alignment cells 76 are configured to be identified to permit positional location determination relative to the sensor 38. For example, an alignment cell 76 may have a known reflectance or attenuation relative to a predetermined wavelength that can be read. In some instances, each alignment cell 76 may have a unique identifier so that it may be distinguished from other alignment cells 76. The present disclosure is not limited to any particular alignment cell 76 configuration. The alignment cells 76 are preferably distributed in a convenient pattern to allow for rapid correction of the steering of the light beams 70, 72, and are preferably located on the perimeter of the Fabry-Perot sensor 38 to avoid interfering with the excitation light beam 72, or reading of the sensing area of the Fabry-Perot sensor 38. The present disclosure device can be configured to perform the alignment automatically without the need for manual and/or mechanical calibration and/or alignment. Routine alignment may be required, for example, due to changes in the device(s) as a result of temperature changes, changes in force applied to the sensor 38, and the like. A significant benefit of the alignment cells 76 is that they can be used when the MEMS mirror 38 is utilized in a resonance mode to facilitate and/or simplify control of the light beam steering. For example, in some instances it may be possible to avoid the need to perform an initial locating procedure (e.g., rastering a light beam solely for locating purposes), and in other instances the alignment cells 76 may be used in an initial locating process and also sensed during operation to confirm and/or adjust positioning.

The controller 26 is in communication with excitation light source 22, the ultrasound receiving mechanism, the light beam steering device 36, and other components within the present disclosure system 20 to perform the functions described herein. The controller 26 may include any type of computing device, computational circuit, processor(s), CPU, computer, or the like capable of executing a series of instructions that are stored in memory. The instructions may include an operating system, and/or executable software modules such as program files, system data, buffers, drivers, utilities, and the like. The executable instructions may apply to any functionality described herein to enable the system 20 to accomplish the same algorithmically and/or coordination of system components. The controller 26 may include a single memory device or a plurality of memory devices. The present disclosure is not limited to any particular type of non-transitory memory device, and may include read-only memory, random access memory, volatile memory, non-volatile memory, static memory, dynamic memory, flash memory, cache memory, and/or any device that stores digital information. The controller 26 may include, or may be in communication with, an input device 78 that enables a user to enter data and/or instructions, and may include, or be in communication with, an output device 80 configured, for example to display information (e.g., a visual display or a printer), or to transfer data, etc. Communications between the controller 26 and other system 20 components may be via a hardwire connection or via a wireless connection. As stated above, the controller 26 may in communication with the excitation laser control unit 52 and/or with the interrogation laser control unit 68, or the aforesaid control units may be integrated with the controller 26.

In the operation of the present disclosure system 20 and method, the sensor head 28 is mounted on the subject in a region where excitation light can access blood vessels to be sensed; e.g., a digit of the subject. As indicated above, a couplant 32 may be disposed between the sensor head 28 and the subject's skin tissue to improve optical signal transmission into the subject's tissue and/or acoustic signal transmission out of the subject's tissue.

In some instances, the present disclosure may include a procedure for aligning and/or controlling scan modes of the light beams 70, 72 relative to the Fabry-Perot sensor 38. Such a procedure may not be necessary in all instances and is therefore an optional aspect of the present disclosure. A non-limiting example of an alignment procedure utilizes a Fabry-Perot sensor 38 having reflectance-encoded alignment cells 76 attached to the Fabry-Perot sensor 38. At least one interrogating light beam is provided to the sensor head 28 and is incident to the light beam steering device 36 (e.g., the two axis MEMS mirror 66). The light beam steering device 36 is controlled to direct the interrogating light beam 70 in a predetermined scan pattern over the Fabry-Perot sensor 38 to ascertain the positions of the reflectance-encoded alignment cells 76; e.g., ascertain orthogonal coordinates (e.g., Xi, Yi, etc.) for each alignment cell 76. Once the alignment cell positions are determined, a steering pattern (e.g., a Lissajous pattern) can be selected based on the alignment cell positions. In some embodiments, the location of the alignment cells 64 may be inferred from a known preset steering pattern (e.g., a steering pattern such as that used during resonant operation), such that corrections for the Fabry-Perot sensor 38 sensitivity can take place periodically or as required by the application at hand.

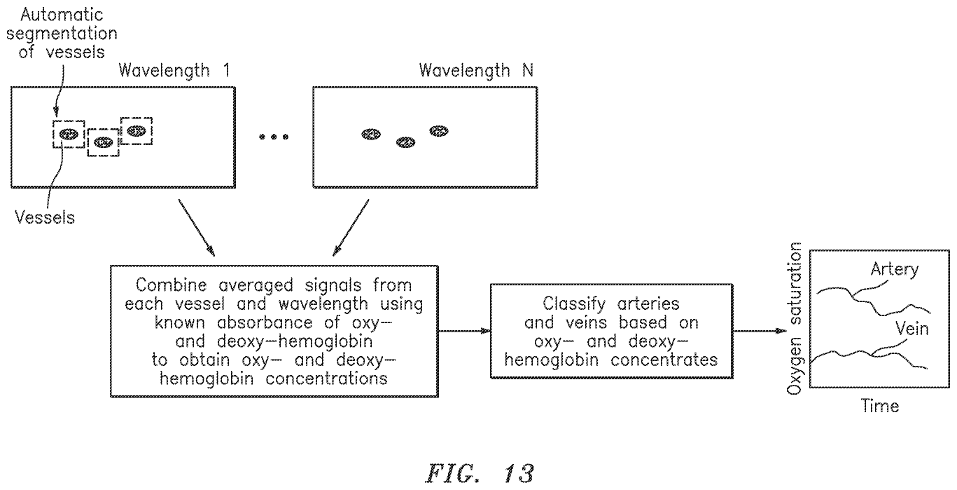

FIG. 12 provides a flow chart depicting an exemplary calibrating procedure that may be used. In this example procedure, initial control parameters are selected for a two axis MEMS mirror 66; e.g., frequency and phase values for actuators disposed within the two axis MEMS mirror 66 (Step 1101). Operation of the MEMS mirror 66 may be initiated using the initial control parameters. In some instances, the procedure may include operating the MEMS mirror 66 for a period of time after start-up to enable the MEMS mirror 66 to reach a stable operating state (Step 1102). Once the MEMS mirror is operating in a stable state, interrogation light reflected from the Fabry-Perot sensor 38 can be collected as described herein for transfer to the light detector 60 and subsequent analysis. More specifically, the interrogation light reflected from the alignment cells 76 (which produce identifiable signals) may be collected as a function of time (Steps 1103, 1104) and analyzed to determine if the control of the MEMS mirror 66 is acceptable (Step 1105). If the light reflected from the alignment cells 76 and/or the timing data indicates that operational adjustment of the MEMS mirror 66 would be beneficial, then adjustments may be made (Step 1106) and the aforesaid process repeated (e.g., return to Step 1102) until the MEMS mirror 66 is satisfactorily operating. As will be discussed below, once the MEMS mirror 66 is satisfactorily operating, the calibrating procedure may include additional steps regarding the sensitivity of the Fabry-Perot sensor 38.

In some embodiments, the present disclosure may include a procedure for calibrating the performance of the ultrasound receiving mechanism. A non-limiting example of such a calibrating procedure includes the determination of a sensitivity map 74 that provides information regarding the uniformity of the photometric response/sensitivity of the sensor 38 to interrogating light beams 70 at a plurality of different wavelengths. The sensitivity map 74 shown in FIG. 11 diagrammatically illustrates sensitivity in terms of orthogonal axes X, Y, Z. The procedure may involve scanning the Fabry-Perot sensor 38 at a low speed in a predetermined pattern a plurality of times (e.g., in an X-Y plane), each time using an interrogating light beam 70 at a different wavelength. The light collected from the Fabry-Perot sensor 38 during this process is analyzed for each interrogation wavelength. The sensitivity map 74 represents an example of a format where the sensitivity data of the respective interrogation wavelengths can be collectively compared (e.g., within the X-Y plane, and along the Z-axis that reflects a parameter such as light intensity, phase, etc.). An optimal wavelength for interrogating the Fabry-Perot sensor 38 may be selected based on this sensitivity data. This process may be repeated periodically or at the user's discretion to account for operational changes (e.g., environmental temperature changes, or a change in the acoustic wave loading of the Fabry-Perot sensor 38, or non-acoustic wave loading, etc.), which operational changes may affect the sensitivity of the Fabry-Perot sensor 38. As indicated above, a Fabry-Perot sensor 38 may be considered to be in a “default” configuration in the absence of acoustic waves acting on the sensor. However, the default configuration may vary as a function of temperature, load/force applied to the sensor 38, etc., which may change the sensitivity of the sensor 38 to a particular interrogation wavelength in a particular area of the sensor. Periodically or when needed, the sensor may be scanned with each interrogating wavelength to create at least one sensitivity map per wavelength. Then, the most sensitive wavelength to a given sensor area is inferred from or by comparing the sensitivity maps. The present disclosure is not limited to using a sensitivity map 74 as part of a calibrating procedure. Referring to FIG. 12, once an interrogating wavelength is chosen, then stability of the sensitivity map 74 can be determined at the chosen interrogating wavelength (Steps 1107-1109). If the stability of the sensitivity map 74 is acceptable, then the stability map 74 may be updated if required. If the stability of the sensitivity map 74 is not acceptable, then process can be repeated until the stability of the sensitivity map 74 is acceptable.

Once any initial procedures that may be required are performed, the present disclosure system 20 can be operated to sense and measure target analytes that may be present within the subject's blood vessel. For example, the controller 26 may execute stored instructions that cause the excitation light source 22 to produce an excitation light beam 72 that includes a particular wavelength of light, and cause the interrogation light source 34 to produce an interrogation light beam 70 that includes one or more wavelengths of light. In some embodiments, the controller 26 may control the excitation light source 22 to vary the wavelength(s) of the excitation light beam 72; e.g., by sequentially operating the respective excitation lasers 40 (e.g., EL1, EL2 . . . ELN) and adding the respective individual light beams into the excitation light beam 72. In some embodiments, the excitation light beam 72 may include light at more than one wavelength. In similar fashion, the controller 26 may control the interrogation light source 34 to vary the wavelength(s) of the interrogation light beam 70; e.g., by sequentially operating the respective interrogation lasers 54 (e.g., IL1, IL2 . . . ILN) and adding the respective individual light beams into the interrogation light beam 72. In some embodiments, the interrogation light beam 70 may include light at more than one wavelength. The controller 26 may execute stored instructions that cause the light beam steering device 36 (e.g., the two axis MEMS mirror 66) to direct the light beams along a selected steering pattern (e.g., a Lissajous pattern) relative to the Fabry-Perot sensor 38. As indicated above, in some embodiments, the excitation and interrogation light beams 72, 70 may be substantially coincident as the light beams traverse the selected steering pattern; e.g., the footprint of the excitation light beam 72, as projected onto the tissue, encompasses the area of the Fabry-Perot sensor 38 that is being read with the interrogating light beam 70. In other embodiments, the interrogation light beam 70 may positionally lag behind the excitation light beam 72 as the light beams traverse the selected steering pattern. In both these embodiments, the interrogation light beam 70 and the excitation light beam 72 are steered together along the selected steering pattern. Once the selected steering pattern is completed, the process may be repeated with the excitation light source 22 producing a light beam at a different wavelength. The process may be repeated as many times as required to capture the desired signal data; e.g., “N” cycles for “N” different excitation wavelengths.

During tissue sensing, the excitation light beams 72 pass through the Fabry-Perot sensor 38 (which is transparent to the excitation light wavelengths) and enter the aligned tissue region. As indicated above, each excitation light wavelength is chosen as one that will produce a photoacoustic effect when light at that wavelength is presented to and absorbed by the target analyte, thereby causing the analyte to produce a response in the form of acoustic waves. At least some of the aforesaid acoustic waves traverse to the skin of the tissue being sensed where they engage with the Fabry-Perot sensor 38. The acoustic waves engaging with the Fabry-Perot sensor 38 cause the sensor 38 to change from its default configuration to a deformed configuration. The interrogating light beam incident to the Fabry-Perot sensor 38 reflects from the sensor 38. The characteristics of the reflected light are a function of the acoustic waves deforming the Fabry-Perot sensor 38 and therefore a function of the photoacoustic effect caused by the target analyte absorbing the excitation light. At least some of the reflected light is collected within the sensor head 28; e.g., received by the objective optics 50, directed to the light beam steering device 36, which in turn directs the collected light to the optical fiber 56 acting as a light conduit between sensor head 28 and the interrogation light source 34. Upon reaching the beam splitter 58 (e.g., see FIG. 4), the beam splitter 58 directs the collected light to the light detector 60. The light detector 60 produces signals representative of the collected light and communicates those signals to the controller 26. The controller 26 is configured to execute algorithmic instructions that cause the controller 26 to determine information such as, but not limited to, the presence of the target analyte, the concentration of the target analyte, etc. based on the signals from the detector 60.

In some embodiments, the controller 26 may include stored instructions that when executed cause the controller 26 to create a vasculature map of the tissue region being sensed. For example, the stored instructions may include an algorithm that uses the signals from the detector 60 to identify a predominant section of blood vessels within the sensed tissue. The type of blood vessel (i.e., an artery or a vein) may be determined using, for example, spectroscopic means. For example, the stored instructions may use the detector signals to determine relative levels of a first analyte (oxyhemoglobin—HbO2) and a second analyte (deoxyhemoglobin—HB) within the blood flow in a given vessel. In some instances, the nature of the vessel (e.g., vein or artery) may be inferred from the relative levels of Hb and HbO2. To give a specific non-limiting example, if the concentration of oxyhemoglobin within the blood flow is above a predetermined level, the blood vessel may be identified as an artery. Conversely, if the concentration of deoxyhemoglobin within the blood flow is above a predetermined level, the blood vessel may be identified as a vein. The present disclosure is not limited to any particular algorithmic steps for identifying vessels and producing a vasculature map therefrom. In some embodiments, the subject tissue may be continuously sensed, and the vasculature map shown in real time. As can be seen from FIG. 13, the algorithm steps may include collecting the detector signals and processing them (e.g., time averaging the signals), determining oxyhemoglobin and deoxyhemoglobin concentrations based on the processed signals, and may include determining physiological/hemodynamic parameters (e.g., oxygen saturation, cardiac output, etc.) based on the determined concentrations.

While various inventive aspects, concepts and features of the disclosures may be described and illustrated herein as embodied in combination in the exemplary embodiments, these various aspects, concepts, and features may be used in many alternative embodiments, either individually or in various combinations and sub-combinations thereof. Unless expressly excluded herein all such combinations and sub-combinations are intended to be within the scope of the present application. Still further, while various alternative embodiments as to the various aspects, concepts, and features of the disclosures—such as alternative materials, structures, configurations, methods, devices, and components, alternatives as to form, fit, and function, and so on—may be described herein, such descriptions are not intended to be a complete or exhaustive list of available alternative embodiments, whether presently known or later developed. Those skilled in the art may readily adopt one or more of the inventive aspects, concepts, or features into additional embodiments and uses within the scope of the present application even if such embodiments are not expressly disclosed herein. For example, in the exemplary embodiments described above within the Detailed Description portion of the present specification, elements are described as individual units and shown as independent of one another to facilitate the description. In alternative embodiments, such elements may be configured as combined elements. As a specific example, in the description above, the controller 26, the excitation light source 22, the interrogation light source 34, and the sensor head 28 are each described as an element and are shown in the Figures as being independent of one another to facilitate the description. The present disclosure contemplates that one or more of these elements may be combined with another element and still be in keeping with the teachings of the present disclosure. For example, in some embodiments the excitation light source 22, the interrogation light source 34, and the controller 26 may be integrated into the sensor head 28.

Additionally, even though some features, concepts, or aspects of the disclosures may be described herein as being a preferred arrangement or method, such description is not intended to suggest that such feature is required or necessary unless expressly so stated. Still further, exemplary or representative values and ranges may be included to assist in understanding the present application, however, such values and ranges are not to be construed in a limiting sense and are intended to be critical values or ranges only if so expressly stated.

Moreover, while various aspects, features and concepts may be expressly identified herein as being inventive or forming part of a disclosure, such identification is not intended to be exclusive, but rather there may be inventive aspects, concepts, and features that are fully described herein without being expressly identified as such or as part of a specific disclosure, the disclosures instead being set forth in the appended claims. Descriptions of exemplary methods or processes are not limited to inclusion of all steps as being required in all cases, nor is the order that the steps are presented to be construed as required or necessary unless expressly so stated. The words used in the claims have their full ordinary meanings and are not limited in any way by the description of the embodiments in the specification.

What is claimed is:

1. A system for non-invasively measuring at least one analyte within a blood vessel, the system comprising:
an excitation light source having at least one excitation laser configured to selectively produce an excitation light beam at a predetermined excitation wavelength, wherein absorption by the analyte of an amount of the excitation light beam causes the analyte to produce a photoacoustic response;
an interrogation light source having at least one interrogation laser configured to selectively produce an interrogation light beam at a predetermined interrogation wavelength;
a Fabry-Perot sensor configured to be transparent to said excitation light beam, and to reflect at least some of the interrogation light beam;
at least one light beam steering device;
a light detector operable to receive light reflected from the Fabry-Perot sensor and produce signals representative of the received light; and
a controller in communication with the excitation light source, the interrogation light source, the at least one light beam steering device, the light detector, and a memory storing instructions, the instructions when executed cause the controller to:
control the light beam steering device to steer both the excitation light beam and the interrogation light beam relative to the Fabry-Perot sensor; and
measure an amount of the analyte within the blood vessel using the signals representative of the received light.

2. The system of claim 1, wherein the system is configured to steer the excitation light beam and the interrogation light beam in unison in a direction of travel along a path relative to the Fabry-Perot sensor.

3. The system of claim 2, wherein the path is a Lissajous pattern.

4. The system of claim 2, wherein the at least one light beam steering device includes a two-axis micro-electro-mechanical (MEMS) mirror, and the instructions when executed cause the controller to control the two-axis MEMS mirror to steer the excitation light beam and the interrogation light beam in unison.

5. The system of claim 4, wherein the instructions when executed cause the controller to control the two-axis MEMS mirror using a resonant excitation.

6. The system of claim 5, wherein the system further comprises a sensor head configured for attachment to a subject, and the two-axis MEMS mirror and the Fabry-Perot sensor are disposed within the sensor head.

7. The system of claim 2, wherein the system is configured to produce the excitation light beam and the interrogation light beam substantially coincident with one another in a sensing area of the Fabry-Perot sensor.

8. The system of claim 2, wherein the system is configured to produce the excitation light beam at a first position on the path, and produce the interrogation light beam at a second position on the path, the second position lagging behind the first position on the path in the direction of travel.

9. The system of claim 2, wherein the at least one light beam steering device includes a first two-axis micro-electro-mechanical (MEMS) mirror and a second two-axis MEMS mirror, and the instructions when executed cause the controller to control the first MEMS mirror to steer the excitation light beam and to control the second MEMS mirror to steer the interrogation light beam in unison.

10. The system of claim 2, wherein the Fabry-Perot sensor has a sensing area, and the system is configured to produce the excitation light source to be incident to the Fabry-Perot sensor in an excitation incident area, and the excitation incident area is less than the sensing area.

11. The system of claim 1, wherein the excitation light source includes a plurality of said excitation lasers, wherein the excitation wavelength produced by each excitation laser is different from the respective excitation wavelength produced by every other of said excitation lasers.

12. The system of claim 11, wherein the instructions when executed cause the controller to operate the excitation lasers sequentially.

13. The system of claim 12, further comprising one or more optical fibers in communication with the excitation light source, the optical fibers configured to accept a plurality of the excitation wavelengths.

14. The system of claim 13, wherein the system is configured so that the light reflected from the Fabry-Perot sensor is received by the one or more optical fibers and passed to a light detector, the light detector configured to produce the signals representative of the received light and communication the signals to controller.

15. The system of claim 1, wherein the at least one interrogation light source includes a plurality of said interrogation lasers, wherein the interrogation wavelength produced by each interrogation laser is different from the respective interrogation wavelength produced by every other of said interrogation lasers.

16. The system of claim 15, wherein the instructions when executed cause the controller to operate the plurality of interrogation lasers sequentially.

17. The system of claim 16, further comprising one or more optical fibers in communication with the interrogation light source, the optical fibers configured to accept a plurality of the interrogation wavelengths.

18. The system of claim 1, wherein the Fabry-Perot sensor includes a plurality of alignment cells, each configured to provide position location information.

19. The system of claim 18, wherein each alignment cell is distinguishable from other said alignment cells by the position location information it is configured to provide.

20. The system of claim 19, wherein the Fabry-Perot sensor has a sensing area, and the plurality of alignment cells are disposed substantially outside the sensing area.

21. The system of claim 1, wherein the instructions when executed cause the controller to calibrate the Fabry-Perot sensor using a sensitivity map.

22. The system of claim 21, wherein the sensitivity map is based on scans of the Fabry-Perot sensor using an interrogation light beam at one or more interrogation wavelengths.

23. The system of claim 1, wherein the instructions when executed cause the controller to create a vascular map of tissue being sensed with the excitation light beam, the vascular map including a location of blood vessels within the tissue.

24. The system of claim 23, wherein the vascular map includes a respective location of one or more veins in the tissue and one or more arteries within the tissue based on relative amounts different analytes sensed within the blood vessels.

25. A method of non-invasively measuring at least one analyte within a blood vessel, the method comprising:
providing a system having an excitation light source with at least one excitation laser configured to selectively produce an excitation light beam at a predetermined excitation wavelength, an interrogation light source having at least interrogation laser configured to selectively produce an interrogation light beam at a predetermined interrogation wavelength, a Fabry-Perot sensor configured to be transparent to said excitation wavelength light, and to reflect at least a portion of the interrogation light beam, at least one light beam steering device, a light detector, and a controller;
using the at least one light beam steering device to steer the excitation light beam and the interrogation light beam in unison in a direction of travel along a path relative to the Fabry-Perot sensor, wherein absorption by the analyte of an amount of the excitation light beam causes the analyte to produce a photoacoustic response;
receiving light reflected from the Fabry-Perot sensor, and using the light detector to produce signals representative of the received light and communicate the signals to the controller; and
measuring an amount of the analyte within the blood vessel using the signals representative of the received light.

26. The method of claim 25, further comprising calibrating the Fabry-Perot sensor using a sensitivity map.

27. The method of claim 26, further comprising scanning the Fabry-Perot sensor using an interrogation light beam at one or more interrogation wavelengths, and producing the sensitivity with light reflected from the Fabry-Perot sensor during the scanning.

28. The method of claim 25, further comprising creating a vascular map of tissue being sensed with the excitation light beam, the vascular map including the location of blood vessels within the tissue.

29. The method of claim 28, wherein the vascular map includes the location of one or more veins in the tissue and one or more arteries within the tissue based on relative amounts different analytes sensed within the blood vessels.
